(19) United States
(12) Patent Application Publication
(10) Pub. No.: US 2018/0305524 A1
(43) Pub. Date: Oct. 25, 2018

(54) Inulin Nanofibers

(71) Applicant: The American University in Cairo, New York, NY (US)

(72) Inventors: Walaa Mohamed Ali Wahbi, Cairo (EG); Wael Mamdouh Sayed Sa Ahmed, Cairo (EG); Rania Siam, Cairo (EG)

(21) Appl. No.: 15/531,087

(22) PCT Filed: Nov. 27, 2015
(86) PCT No.: PCT/US15/62839

§ 371 Date: May 26, 2017

Related U.S. Application Data
(60) Provisional application No. 62/085,395, filed on Nov. 28, 2014.

(51) Int. Cl. C08L 5/00; C08L 29/04; A61L 26/00; A61K 47/32; A61K 47/36; A61K 31/765; A61K 31/733; A01N 43/16; A01N 31/02; A01N 25/34; A61K 9/00; D01D 5/00; D01F 1/10; D01F 6/14; D01D 10/02; D01F 11/06

(52) U.S. Cl. CPC C08L 5/00; D10B 2509/00; A61L 26/0023; A61L 26/0014; A61L 26/009; A61K 47/32; A61K 47/36; A61K 31/765; A61K 31/733; A01N 43/16; A01N 31/02; A01N 25/34; A61K 9/0092; D01D 5/003; D01F 1/10; D01F 6/14; D01D 10/02; D01F 11/06; C08L 2205/16; A61L 2300/232; A61L 2400/12; C08L 2203/02; D10B 2321/06; D10B 2509/022; D10B 2401/13; D10B 2401/12; C08L 29/04

(57) ABSTRACT

Electrospun Polyvinyl Alcohol (PVA)/Inulin composite nanofibers (CNFs) are provided using electrospinning technique and tested for their prebiotic and antibacterial activities. The PVA/Inulin electrospun CNFs were tested for prebiotic activity with Lactobacillus sp. and for antibacterial activity against both Escherichia coli (E. coli) and Staphylococcus aureus (S. aureus). A number of electrospinning parameters such as solution concentration, PVA: Inulin mixing ratio, solution flow rate and applied voltage were carefully varied and the best PVA/Inulin electrospun CNFs (bead free) were selected for prebiotic and antibacterial tests. The concentration of the composite solution varied between 14-20%, the flow rate ranged between 0.005-0.5 mL/min and the applied voltage used ranged between 15-20 Kv. The structural properties and morphology of the PVA/Inulin electrospun CNFs were fully characterized by Fourier Transform Infrared Spectroscopy (FT-IR) and scanning electron microscopy (SEM).

### FIELD OF THE INVENTION

This invention relates to prebiotics and antibacterial assessment. In particular, the invention relates to inulin nanofibers.

### BACKGROUND OF THE INVENTION

The human gastrointestinal microbiota plays an important role in improving human health and preventing different gut diseases. The microbiota's function is to prevent and/or reduce pathogenic bacteria colonization. The gastrointestinal tract (GIT) is inhabited by a complex community of microorganisms and the large intestinal microbiota only is inhabited by more than 400 bacterial species with bacterial population compromising approximately 10¹¹-10¹² cfu/gm of colonic contents but lactobacilli and bifidobacteria are the most predominant. The main dietary materials that contribute to the growth of the large intestinal microbiota are carbohydrate-based materials while nitrogen-based materials like proteins show less contribution. Carbohydrates that resist hydrolysis and absorption in small intestine support the growth of the intestinal bacterial population. Probiotics, prebiotics and synbiotics are the main dietary components that may modulate the flora.

The composition of the large intestinal microbiota is affected by several factors including age, presence of fermentable compounds in the gut and the use of antibiotics. The intestinal microbiota has been associated with various disturbances due to small intestinal bacterial overgrowth or antibiotic-associated diarrhea, gastroenteritis, and irritable bowel syndrome (IBS). Silk et al. reported that galactooligosaccharides were effective as a prebiotic in IBS patients, where the gut bifidobacteria was stimulated and the symptoms were improved.

Furthermore, Thoua et al. reported on several assessments that showed the beneficial effect of probiotics in IBS symptom.

Consequently, using prebiotics to control intestinal microbiota and alleviate GIT disorders are of great interest these days. Increasing lactobacilli and bifidobacteria, known as probiotics, has been responsible for the beneficial effects that take place in the human gut⁶.

Probiotics are living organisms that exert health benefits to the host when ingested in adequate amounts. Probiotic bacteria produce lactic acid as the major end product of the fermentation of carbohydrates. Currently, the most common probiotics used belong to lactobacillus and bifidobacterium genera.

The beneficial effects of probiotics originate from lowering the intestinal pH due to fermentation of carbohydrates, which result in the formation of short chain fatty acids (SCFA), suppression of pathogenic bacteria, and stimulation of immune system.

Due to the fact that people have doubts about consuming live bacteria, probiotics don't function as desired in absence of prebiotics and probiotics stability is affected by manufacturing, storage and GIT conditions; prebiotics have attracted attention. The world demand for prebiotics is estimated to be around 167,000 tons and 390 million Euros.

Prebiotics are compounds, usually polysaccharides and oligosaccharides, which are resistant to metabolism and reach the intestine to be utilized by beneficial bacteria. They may occur naturally in some foods such as chicory, Jerusalem artichokes, garlic, onion, dahlia tubers and others.

Prebiotics are resistant to enzymatic hydrolysis in the upper GIT but they are fermented completely in the large intestine to produce lactate, short chain fatty acids (SCFA) such as acetate, butyrate and propionate, and gases. These resultant acids lower the intestinal pH, which consequently results in a decrease in the number of pathogenic bacteria.

The aim of supplementing human diet with prebiotic oligosaccharides is the beneficial modulation of the human gut microbiota by stimulating endogenous beneficial gut bacteria and suppressing pathogenic bacteria.

Currently, great interest in using natural products has been revealed due to the development of drug resistance infections and the demand for functional food. Natural polysaccharides obtained from different sources have greatly attracted the biomedical field attention due to their low toxicity and therapeutic activity broad spectrum.

Inulin is one of the important natural products with great interest as a prebiotic. It is a naturally occurring storage carbohydrate found in plants such as chicory, Jerusalem artichoke, and dahlia tubers.

Due to the specific beta linkage between fructose monomers, inulin resists enzymatic hydrolysis by human salivary and small intestine digestive enzymes, and reaches colon unchanged where they are fermented by intestinal microbiota to be converted into short chain fatty acids, lactate and gases.

Researchers have studied the prebiotic activity of inulin. López-Molina et al. studied the prebiotic effectiveness of inulin extracted from artichoke on Bifidobacterium bifidum culture. Inulin increased the growth of B. bifidum, confirming the effectiveness of inulin as a prebiotic.

Pompei et. al examined the prebiotic activity of an oligofructose (OF) and inulin in vitro, and both showed clear prebiotic effectiveness. However, the increase in bifidobacteria and lactobacillus concentrations occurred earlier in oligofructose, and this was attributed to the short chains of OF which are easily metabolized compared with longer chains of inulin.

The present invention provides a method for synthesis and utilization of electrospun nanofibers using inulin. With respect to the use, since the nano-scaled materials exhibit different properties compared to their bulk form, this invention also entails our study of the effect of inulin nanofibers on their prebiotic and antibacterial activities.

Among the different types of nanomaterials, nanofibers have attracted a lot of attention in various fields due to their large surface areas per unit mass and advanced mechanical performance which makes them potential candidates to be used in catalysis, drug loading, and etc.

There are various methods for fabricating nanofibers such as drawing, template synthesis, self-assembly, phase separation and electrospinning. Although there are various techniques for nanofiber synthesis, however, electrospinning is the most popular and attractive technique for the fabrication of nanofibers.

Due to the limitation of natural polymers, synthetic polymers are more widely used and they are tailored to fabricate nanofibers of desired properties. They can be electrospun alone or following their combination with other polymers either natural or synthetic. Synthetic polymers include polyvinyl alcohol (PVA), polyethylene oxide (PEO), poly (latic acid-co-glycolic acid) (PLGA), and polylactide (PLA).

L. M. M. Costa et al. used PVA to develop a nanocomposite of pineapple nanofibers with Stryphnodendron adstringens bark extract by electrospinning. Before using the electrospun nanofibers in medical implants, they need to be assessed for their toxicity.

Wang H et al. prepared PVA/oxidized starch nanofibers with different solution concentrations by electrospinning and the nanofibers were characterized. The PVA/OS nanofibers can be used in drug delivery wound dressing material due to biodegradability and lack of toxicity.

### SUMMARY OF THE INVENTION

The present invention provides PVA/Inulin composite nanofibers to have applications in wound dressings, drug delivery, surface coatings, antiseptic sprays and in treatment of digestive disorders.

Polyvinyl Alcohol (PVA)/Inulin nanofibers are manufactured using electrospinning technique and tested for their prebiotic activity with Lactobacillus sp. and antibacterial activity with E. coli and S. aureus. After characterization and cross-linking of the produced PVA/Inulin Electrospun composite nanofibers (CNF), they were tested for their prebiotic activity with Lactobacillus sp. by viable count, optical density, pH and growth curve, and antibacterial activity with E. coli and S. aureus, by the cork-borer method, measuring the inhibition zone and the inhibition curve. Interestingly, the PVA/Inulin electrospun CNF showed an increase in the lactobacillus growth from 2.9×10³ cfu/mL (with respect to inulin solution) to 4.0×10³ cfu/mL (i.e. increased by 37.9%), and the growth curve showed that the growth of the culture containing PVA/Inulin electospun CNFs is not substantially greater than the growth of the control.

On the other hand, the inulin solution itself didn't show any visible zone of inhibition with both E. coli and S. aureus. Surprisingly, PVA/Inulin electrospun CNFs exhibited inhibition zones of 18.3 mm with both E. coli and S. aureus. The inhibition curve showed that there was decrease in the growth of S. aureus, and a slight decrease of E. coli.

This shows the unique prebiotic and antibacterial effects of the nanoscale transformation of inulin solution versus inulin nanofibers.

In one embodiment, a composition of electrospun composite nanofibers is provided. The composite nanofibers are cross-linked polyvinyl alcohol (PVA) and inulin electrospun nanofibers. The inulin is in the range of 4 to 10% of the total weight of the composite nanofibers. The PVA is 8% to 12%, preferably at 10%, of the total weight of the composite nanofibers. The composite nanofibers are produced at a range of 300 nm to 640 nm. The composite nanofibers are chemically crosslinked by glutaraldehyde. Electrospinning parameters for the composite nanofibers are high voltages between 16-20 kv and flow rates of 0.005-0.5 mL/min. In one example, biocompatible synthetic polymers can be used either alone or in combination: PEO (polyethylene glycol), PLA (polylactic acid), PLLA (poly-L-lactic acid), PET (polyethylene terephthalate), and PP (polypropylene). In another example, curcumin (turmeric), alovera oil or extract, olive oil, garlic, garlic extract, olive extract or chamomile, apple cidar vinegar, honey can be added to the nanofibers. In still another example, gelatin, collagen, alginate, chitosan can be added to the nanofibers. In yet another example, bacteriophage, bee venom, beeswax, enzymes can be added to the nanofibers. In yet another example, oxacillin, ciprofloxacin or penicillin can be added to the nanofibers. In yet another example, the lectrospun nanofibers are produced on static and can be produced moving collector. In yet another example, the electrospun nanofibers can be produced at room temperature or at a temperature above room temperature. In yet another example, the electrospun composite nanofibers are crosslinked physically by thermal treatment. The electrospun nanofibers can be used in wound dressing, treatment of digestive disorders, antiseptic sprays, surface nano-coatings inside hospitals, sterile areas and pharmaceutical facilities.

### BRIEF DESCRIPTION OF THE DRAWINGS

FIGS. 1A-C show according to an exemplary embodiment of the invention in FIGS. 1A-B SEM images of PVA/Inulin electrospun CNFs fabricated from 15% (w/w) blend solution at voltage of 16 kv and flow rate of 0.1 mL/min. Corresponding histogram showing the fiber diameter distribution (FIG. 1C).

FIGS. 2A-C show according to an exemplary embodiment of the invention in FIG. 2A Total viable count, FIG. 2B pH, and FIG. 2C Optical density of Lactobacillus sp. culture containing PVA electrospun Nanofibers, inulin solution, PVA/Inulin electrospun CNFs, and water after 24 hours of incubation.

FIG. 3 shows according to an exemplary embodiment of the invention growth curve of Lactobacillus sp. culture containing PVA/Inulin electrospun CNFs.

FIGS. 4A-B show according to an exemplary embodiment of the invention in FIG. 4A an inhibition zone of PVA electrospun Nanofibers, inulin solution, PVA/Inulin electrospun CNFs, antibiotic and water with E. coli, in FIG. 4B an inhibition zone of PVA electrospun Nanofibers, inulin solution, PVA/Inulin electrospun CNFs, antibiotic and water with S. aureus.

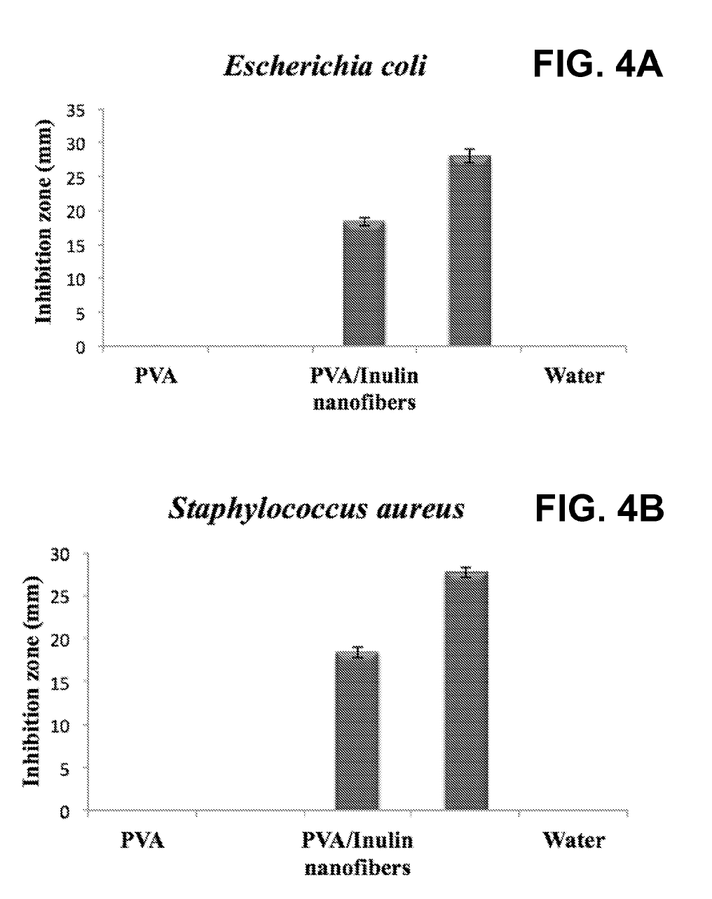

FIGS. 5A-B show according to an exemplary embodiment of the invention an inhibition curve of E. coli (FIG. 5A) and S. aureus (FIG. 5B) culture of PVA/Inulin electrospun CNFS (Optical density).

### DETAILED DESCRIPTION

The objective of this invention is the fabrication of nanofibers of Inulin, a naturally occurring polysaccharide, with enhanced prebiotic and antibacterial activities. The description is divided into:
(i) Fabrication of electrospun composite nanofibers (CNFs) from Inulin and Poly vinyl alcohol (PVA) using an electrospinning technique,
(ii) Characterization of the electrospun CNFs (morphological by scanning electron microscopy (SEM) and spectroscopically by Fourier Transform Infrared (FT-IR) Spectroscopy),
(iii) Cross-linking the successfully electrospun CNFs by physical and chemical methods to select the most efficient cross-linking method that could keep the mesh structures of the fabricated CNFs when dissolved in other solvents, and
(iv) Testing the electrospun CNFs (both cross-linked and non cross-linked) for their prebiotic and antibacterial activities.

In the first set of CNFs fabrication experiments by the Electrospinner, Inulin couldn't be directly electrospun into uniform nanofibers at any concentration, except after mixing with PVA polymer to improve the spinning ability of inulin. A wide variety of concentrations of the PVA/Inulin blend solutions of (14, 15, 16, 18 &20) % w/w were prepared. In addition, applied voltages of (16-20 kv) and flow rates of (0.005 to 0.5 mL/min.) were used.

Nanofibers electrospun from PVA/Inulin blend solution of concentration 15% w/w at voltage 16 kv and flow rate 0.1 mL/min were selected to be the best parameters to produce smooth, uniform and beads-free nanofibers.

The electrospun CNFs have been tested for their prebiotic and antibacterial with three types of bacteria: Lactobacillus sp., gram positive and gram negative (E. coli and S. aureus bacteria).

To the best of our knowledge, this is the first study to report on the successful fabrication of electrospun nanofibers using inulin and more importantly testing the fabricated CNFs for their prebiotic and antibacterial with three types of bacteria. The parameters of the electrospinning such as the applied voltage, concentration of the PVA/Inulin blend solution as well as the flow rate were varied and adjusted to give the most acceptable PVA/Inulin electrospun CNFs. The choice of PVA as the main polymer to be mixed with inulin was mainly due to its chemical stability at room temperature along with its unique physical properties, which made it one of the most acceptable polymers that is mainly used in fiber fabrication.

### Preparation of PVA/Inulin Electrospun CNFs

A wide variety of concentrations of the PVA/Inulin blend solutions of (14, 15, 16, 18 &20) % w/w were prepared. The concentration of PVA was kept constant (10 grams) in all samples, and the inulin concentration was varied between 4-10 grams to obtain PVA: Inulin ratios between 2.5:1 to 1:1, leading to total mixture concentration of 14-20 (w/w %). PVA aqueous solutions were prepared by weighing PVA in distilled water. Then the solutions were stirred with a magnetic stirrer at temperatures ≤100° C. for a period of less than 90 min to acquire a homogenous solution. Inulin aqueous solutions were prepared by dissolving inulin in distilled water at room temperature. Then aqueous solutions of PVA and inulin were added to either to obtain blend solution of PVA/Inulin.

### Electrospinning Parameters of PVA/Inulin CNFs

Electrospinning was carried out by using a commercial electrospinner (E-Spin Tech, India) with a syringe pump and a high voltage power supply (Gamma High Voltage power supply, USA). The solutions were loaded into a plastic syringe connected to a sharp tip needle, which was grounded by a crocodile clip.

Electrospinning parameters were adjusted as follows; high voltages 16-20 kv and flow rates of 0.005-0.5 mL/min. Aluminum foil sheets were used to cover copper plate collector, and the distance from the tip of the needle to the collector was adjusted to 10 cm. Electrospinning of all solutions mixtures were carried out at room temperature. PVA/Inulin electrospun CNFs were successfully produced by electrospinning using 15% w/w blend solution at 16 kv applied voltage and a flow rate 0.1 mL/min.

The parameters for producing PVA/Inulin electrospun CNFs were:
Effect of solution concentration on the morphology of PVA/Inulin electrospun CNFs. To observe the changes in fiber formation, and to select the parameters that produce smooth, uniform and beads-free CNFs.
Effect of applied voltage on the morphology of PVA/Inulin electrospun CNFs. To observe the changes in nanofiber formation and nanofibers morphology upon varying the applied voltage. And to select the parameters that produce CNFs with desired morphology.
Effect of solution flow rate on the morphology of PVA/Inulin electrospun CNFs. To observe the changes in nanofiber formation and nanofibers morphology upon varying the flow rate. And to select the parameters that produce CNFs with desired morphology.

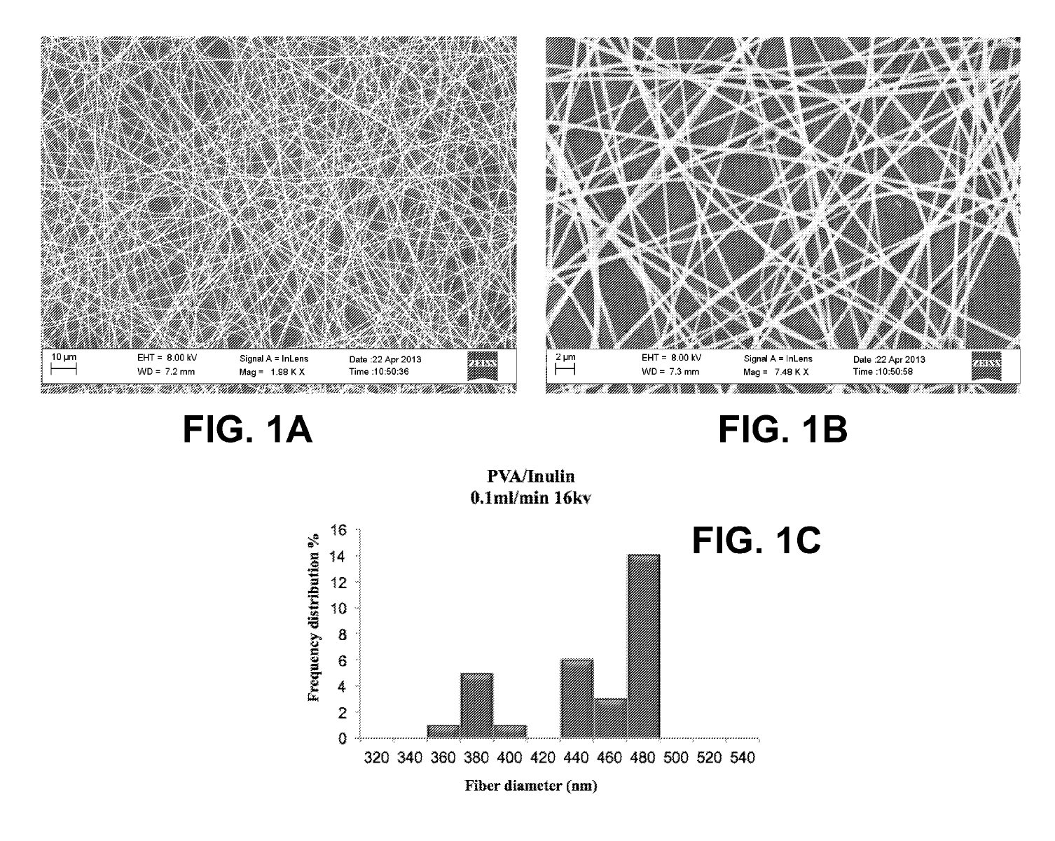

FIGS. 1A-B show SEM images of PVA/Inulin electrospun CNFs fabricated after a number of assessments to accomplish the most acceptable electrospinning parameters.

### Cross-Linking of PVA/Inulin CNFs

Physical and chemical cross-linking of the PVA/Inulin electrospun CNFs (smooth, uniform and bead free) were carried out to obtain the most reliable cross-linking method.

1. Physical Cross-Linking

Physical cross-linking was performed by thermal treatment of the electrospun PVA/Inulin CNFs in a vacuum oven (Jelotech, OV-11, Korea) at temperatures from 80° C. to 140° C. for 10 minutes.

2. Chemical Cross-Linking

Glutaraldehyde solution (GA) was used for chemical cross-linking of the PVA/Inulin electrospun CNFs. The electrospun CNFs were placed inside a desiccator occupied with the vapors of 50 mL of GA solution. Exposure time to GA vapor varied from 30 to 120 minutes and then were thermally treated for 24 hours in an oven at 70° C. under vacuum.

### Water Immersion Test

To investigate the efficiency of both cross-linking methods on the PVA/Inulin electrospun CNFs, the stability of the electrospun CNFs in warm distilled water at 37° C. for 24 hours was tested. Then the electrospun CNFs were immediately weighed after removing the surface water with filter paper. The weight of the dry cross-linked composite nanofibers was calculated by determining the weight loss according to equation (1). The weight before immersion in water (wᵢ) and after immersion in water and drying (w_{f}) were measured.

$$wt \cdot loss \% = \frac{{w}_{i} - {w}_{f}}{{w}_{i}} \times 100 \qquad ( 1 )$$

Prebiotic Activity

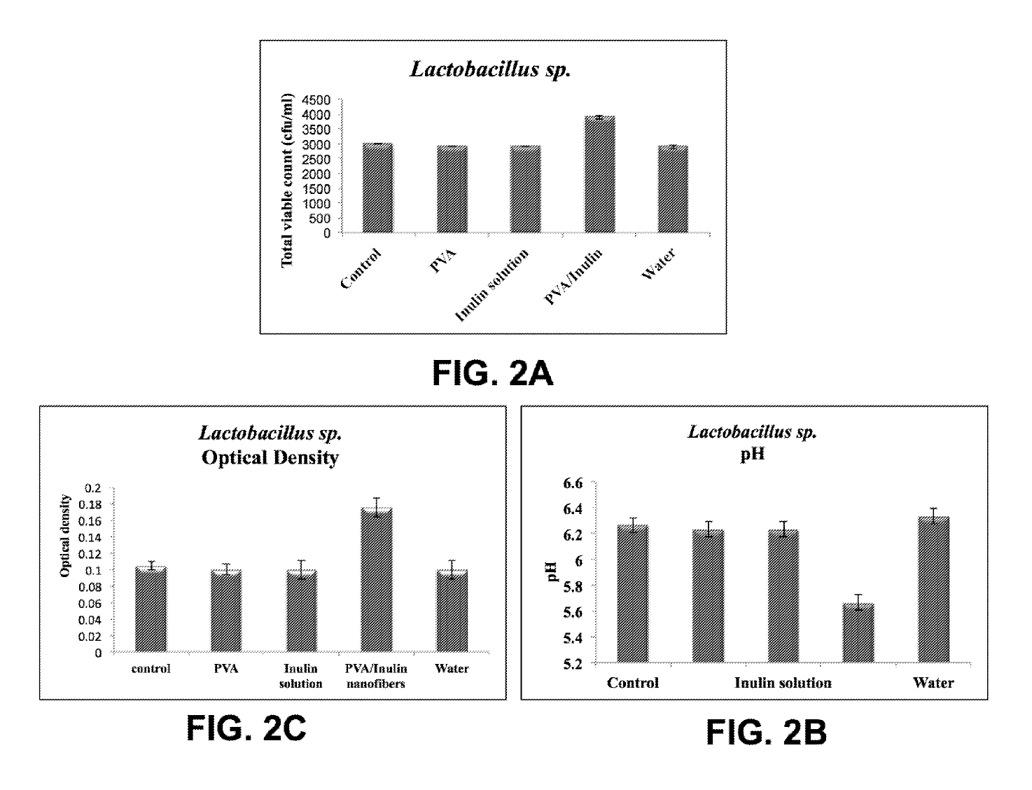

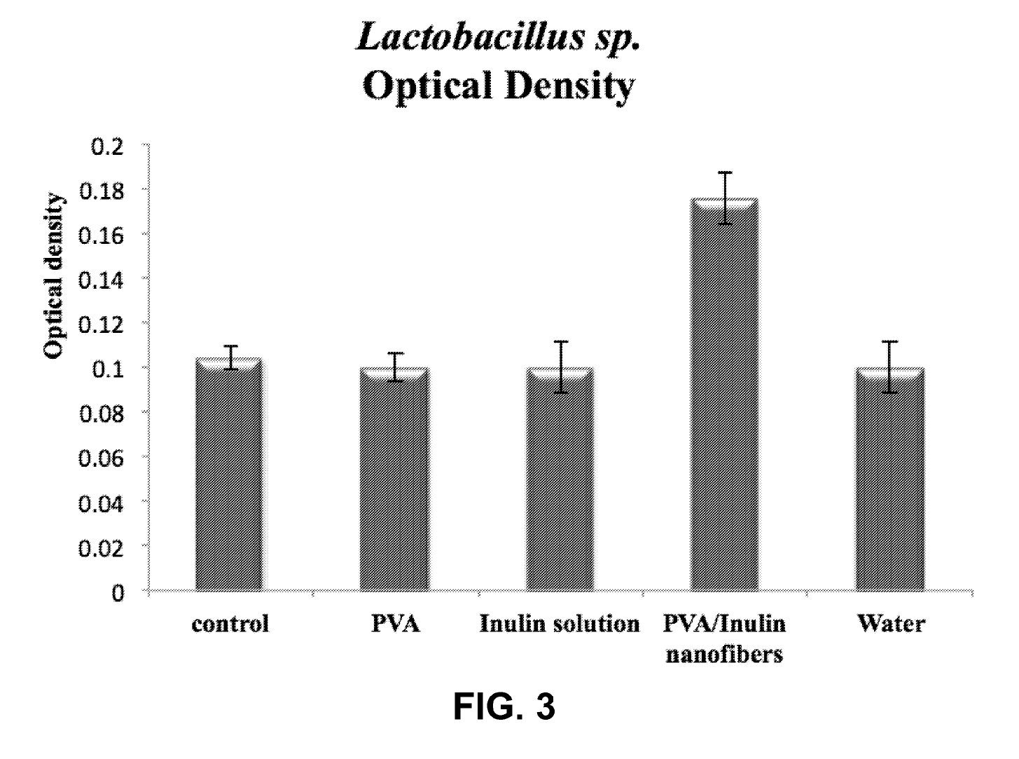

Prebiotic activity was carried out using Lactobacillus sp. to assess the growth activity by calculating the i) total viable counts, ii) pH, iii) optical density (OD), and iv) growth curve. The results of the prebiotic activity of the PVA/Inulin electrospun CNFs showed an increase in the lactobacillus growth from 2.9×10³ cfu/mL (with inulin) to 4.0×10³ cfu/mL (increased by 37.9%) (FIGS. 2A-C). The inhibition curve showed that there was decrease in the growth of S. aureus, and a slight decrease of E. coli (FIG. 3).

The pH and the OD of the culture inoculated with the tested material were measured before incubation and after 24 hours incubation. PVA/Inulin electrospun CNFs solution decreased the pH to 5.7 compared to 6.3 for the control. The inulin solution showed no decrease in the pH, and remained at 6.2 (FIG. 2B). Additionally, the PVA/Inulin electrospun CNFs recorded the highest OD reading, following 24 hours incubation, among the tested samples (FIG. 2C). The results indicate that the PVA/Inulin electrospun CNFs exhibited higher prebiotic activity than inulin solution alone.

In FIG. 3, the growth curve showed that the growth of the culture containing PVA/Inulin electospun CNFs is not substantially greater than the growth of the control.

Antibacterial Activity

To the best of our knowledge, the antibacterial activity of inulin hasn't been previously reported. The antibacterial activity of prebiotics generally and inulin particularly occurs only after their fermentation by the probiotics. Fermentation of the prebiotics produces short chain fatty acids that decrease the pH of the gut environment, which the pathogenic bacteria can't tolerate.

Water, PVA electrospun Nanofibers and inulin solution didn't show any visible zone of inhibition with both E. coli and S. aureus. This confirms that they don't exhibit antibacterial activity. Surprisingly, PVA/Inulin electrospun CNFs showed high antibacterial activity with E. coli and S. aureus compared with inulin solution. The PVA/Inulin electrospun CNFs exhibited inhibition zone of 18.3 mm with both E. coli and S. aureus. On the other hand, inulin solution didn't exhibit any inhibition zone with both E. coli and S. aureus. This shows the unique antibacterial effect of the nanoscale transformation of inulin solution versus inulin nanofibers.

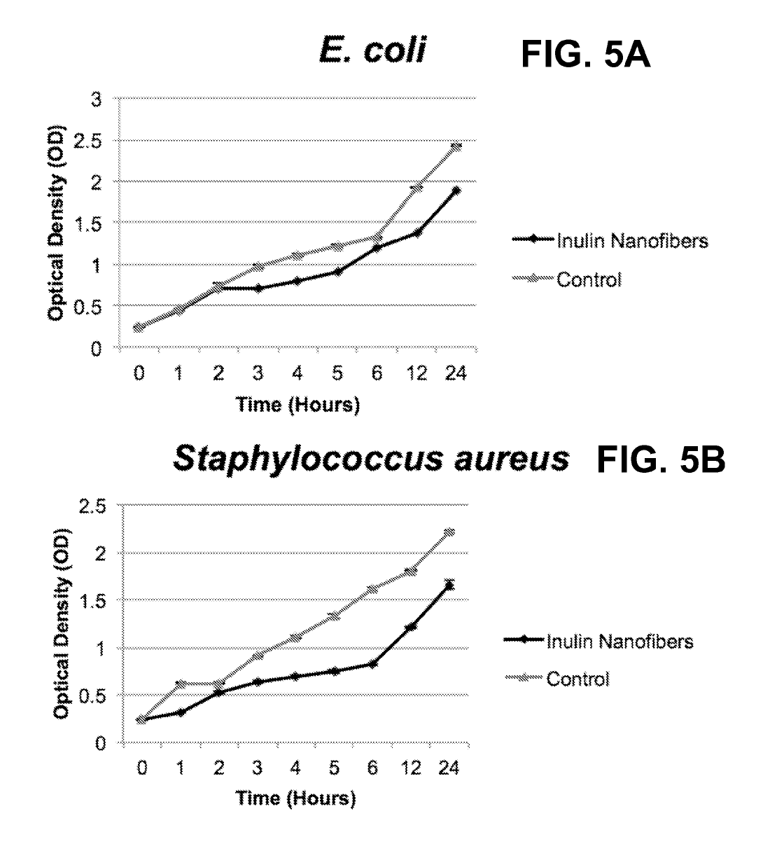

The inhibition curve showed that there was decrease in the growth of S. aureus in FIG. 5b, and a slight decrease of E. coli in FIG. 5A. The growth of the culture containing PVA/Inulin electospun CNFs with S. aureus is significantly less than the growth of the control. This confirms the antibacterial activity of PVA/Inulin electospun CNFs with S. aureus.

From the results presented, we concluded that PVA/Inulin electrospun CNFs possess an enhanced prebiotic activity. Moreover unlike inulin, the PVA/Inulin electrospun CNFs possess antibacterial activity with both Gram-negative E. coli and Gram-positive S. aureus.

One of the main reasons behind the enhanced prebiotic and antibacterial activities of the electrospun composite nanofibers is ascribed to the increased surface area to volume ratio of the electrospun nanofibers available for interaction with bacteria. These results are in agreement with the results reported by L. Qi et al., similarly reporting that chitosan nanoparticles exhibited higher antibacterial activity than chitosan due to the larger surface area of chitosan nanoparticles. Qi, L.; Xu, Z.; Jiang, X.; Hu, C.; Zou, X. Carbohydr. Res. 2004, 339, 2693-2700.

### Advantages

The composite nanofibers are expected to possess many advantages compared to their original non-electrospun solutions. The advantages of our electrospun CNFs are mainly: (i) their enhanced prebiotic activity, and (ii) enhanced antibacterial activity, which are directly related to the large surface area per unit mass of the fabricated electrospun composite nanofibers, and the availability of more binding sites on their surfaces towards the two types of bacteria.

Electrospun CNFs are mainly composed of natural materials, which are not harmful for your human consumption, and offer enhanced prebiotic and antibacterial activity with minimal use of synthetic chemicals compared to their non-electrospun solutions. These nanofibers have a wide variety of possible applications against different types of bacteria.

Uses

The electrospun CNFs could be used for the treatment of digestive disorders, antiseptic sprays or bandages' fillers for wound infections, and many different types of bacterial infections. These electrospun CNFs could also be used as surface nano-coatings inside hospitals, sterile areas and pharmaceutical facilities.

### REFERENCES

(1) Saad, N.; Delattre, C.; Urdaci, M.; Schmitter, J. M.; Bressollier, P. LWT—Food Sci. Technol. 2013, 50, 1-16.
(2) Tuohy, K. M.; Probert, H. M.; Smejkal, C. W.; Gibson, G. R. Drug Discov. Today 2003, 8, 692-700.
(3) Slavin, J. Nutrients 2013, 5, 1417-1435.
(4) Quigley, E. M. M. Pharmacol. Res. 2010, 61, 213-218.
(5) Ito, M.; Deguchi, Y.; Miyamori, a.; Matsumoto, K.; Kikuchi, H.; Kobayashi, Y.; Yajima, T.; Kan, T. Microb. Ecol. Health Dis. 1990, 3, 285-292.
(6) Gibson, G. R.; Roberfroid, M. B.; J. Nutr. 1995, 125, 1401-1412.
(7) Collins, M. D.; Gibson, G. R. Am. J. Clin. Nutr. 1999, 69, 1052S-1057S.
(8) Quigley, E. M. M. Nutr. Clin. Pract. 2012, 27, 195-200.
(9) Silk, D. B. A; Davis, A; Vulevic, J.; Tzortzis, G.; Gibson, G. R. Aliment. Pharmacol. Ther. 2009, 29, 508-518.
(10) Thoua, N. M.; Murray, C. D. Medicine (Baltimore). 2011, 39, 214-217.
(11) Kolida, S.; Tuohy, K.; Gibson, G. R. Br. J. Nutr. 2007, 87, 5193.
(12) Liong, M. T. Int. J. Mol. Sci. 2008, 9, 854-863.
(13) Mussatto, S. I.; Mancilha, I. M. Carbohydr. Polym. 2007, 68, 587-597.
(14) Gibson, G. R.; Scott, K. P.; Rastall, R. A.; Tuohy, K. M.; Hotchkiss, A., Dubert-Ferrandon, A.; Gareau, M.; Murphy, E. F.; Saulnier, D.; Loh, G.; Macfarlane, S.; Delzenne, N.; Ringel, Y.; Kozianowski, G.; Buddington, R.; Lenoir-Wijnkoop, I.; Walker, C. Food Sci. Technol. Bull. Funct. Foods 2010, 7, 1-19.
(15) Azmi, A. F. M. N.; Mustafa, S.; Hashim, D. M.; Manap, Y. A. Molecules 2012, 17, 1635-1651.
(16) Niness, K. R. J. Nutr. 1999, 129, 1402S-1406S.
(17) Singh, G.; Kumar, P. Indian J. Pharm. Sci. 2011, 73, 473-478.
(18) Schepetkin, I.; Quinn, M. T. Int. Immunopharmacol. 2006, 6, 317-333.
(19) Roberfroid, M. J. Nutr. 2007, 137, 830S-7S.
(20) Pompei, A.; Cordisco, L.; Raimondi, S.; Amaretti, A.; Pagnoni, U. M.; Matteuzzi, D.; Rossi, M. Anaerobe 2008, 14, 280-286.
(21) López-Molina, D.; Navarro-Martínez, M. D.; Rojas Melgarejo, F.; Hiner, A. N. P.; Chazarra, S.; Rodríguez-López, J. N. Phytochemistry 2005, 66, 1476-1484.
(22) Tan, S.; Huang, X.; Bolin W. Polym. Int. 2007, 56, 1330-1339.
(23) Ramakrishna, S.; Fujihara, K.; Teo, W.; Yong, T.; Ma, Z.; and Ramaseshan, R. Mater. today 2006, 9, 40-50.
(24) Koski, A.; Yim, K.; Shivkumar, S. Mater. Lett. 2004, 58, 493-497.
(25) Jia, L.; Qin, X. J. Therm. Anal. calorim. 2012, 112, 595-605.
(26) Li, N.; Qin, X.; Yang, E.; Wang, S. Mater. Lett. 2008, 62, 1345-1348.
(27) Fong, H.; Chun, I.; Reneker, D. Polymer (Guildj). 1999, 40, 4585-4592.
(28) Khalil, K. A.; Fouad, H.; Elsarnagawy, T.; Almajhdi, F. N. Int. J. Electrochem. Sci., 2013, 8, 3483-3493.
(29) Vasita, R.; Katti, D. S. Int. J. Nanomedicine 2006, 1, 15-30.
(30) Costa, L. M. M.; de Olyveira, G. M.; Cherian, B. M.; Leão, A. L.; de Souza, S. F.; Ferreira, M. Ind. Crops Prod. 2013, 41, 198-202.
(31) Wang, H.; Wang, W.; Jiang, S.; Zhai, L.; Jiang. Q. Iran. Polym. J. 2011, 20, 551-558.
(32) Kang, Y. O.; Yoon, I.-S.; Lee, S. Y.; Kim, D.-D.; Lee, S. J.; Park, W. H.; Hudson, S. M. J. Biomed. Mater. Res. B. Appl. Biomater. 2010, 92, 568-576.

What is claimed is:

1. A composition of electrospun composite nanofibers, comprising cross-linked polyvinyl alcohol (PVA) and inulin electrospun nanofibers, wherein the inulin is 4 to 10% of the total weight of the composite nanofibers.

2. The composition as set forth in claim 1, wherein the PVA is 8% to 12% of the total weight of the composite nanofibers.

3. The composition as set forth in claim 1, wherein the composite nanofibers are produced at a range of 300 nm to 640 nm.

4. The composition as set forth in claim 1, wherein the composite nanofibers are chemically crosslinked by glutaraldehyde.
